(19) United States
(12) Patent Application Publication
(10) Pub. No.: US 2025/0132062 A1
(43) Pub. Date: Apr. 24, 2025

(54) DETERMINING MISSING DATA CLASSIFICATIONS BY CORRELATING PREDICTION OUTPUTS GENERATED BY MACHINE LEARNING PREDICTIVE SYSTEMS

(71) Applicant: Optum Services (Ireland) Limited, Dublin (IE)

(72) Inventors: Michael J. McCarthy, Dublin (IE); Neill Michael Byrne, Dublin (IE)

(21) Appl. No.: 18/404,412

(22) Filed: Jan. 4, 2024

Related U.S. Application Data
(60) Provisional application No. 63/591,786, filed on Oct. 20, 2023.

(51) Int. Cl. G16H 70/60; G06N 5/022

(52) U.S. Cl. CPC G16H 70/60; G06N 5/022

(57) ABSTRACT

Various embodiments of the present disclosure provide methods, apparatus, systems, computing devices, computing entities, and/or the like for generating a correlated prediction for an input data record by generating a correlation matrix based on co-occurrences associated with a plurality of reference non-correlated predictions, generating a simulation matrix comprising a plurality of simulation data records based on a number of simulation instances and the plurality of reference non-correlated predictions, generating a plurality of correlated simulation data records based on the correlation matrix and select ones of the plurality of simulation data records, generating one or more univariates based on the plurality of correlated simulation data records, and determining a correlated prediction based on a comparison of the one or more univariates and a plurality of input non-correlated probabilities associated with the input data record.

### CROSS REFERENCE TO RELATED APPLICATION

This application claims the priority of U.S. Provisional Application No. 63/591,786, entitled “GENERATING SINGLE SUSPECT RISK SCORE FROM CORRELATED INDIVIDUAL DISEASE RISK SCORES,” filed on Oct. 20, 2023, the disclosure of which is hereby incorporated by reference in its entirety.

### BACKGROUND

Various embodiments of the present disclosure address technical challenges related to data prediction and imputation, generally, and, more specifically, provide solutions to address technical shortcomings with existing multi-label classification solutions.

Deriving predictions based on machine learning or other methods may be used in a plurality of multi-label classification domains, such as in the healthcare industry in which various categories may be used to facilitate healthcare services. For instance, a membership base may be analyzed, using multi-label classification techniques, to generate prediction scores for various disease categories that may inform treatments across the membership base. In practice, these disease categories may be documented using International Classification of Diseases (ICD) codes that together make up a multi-label classification problem. That is, each particular code may require individualized prediction techniques to evaluate an individual's association with that particular code such that, to sufficiently analyze each code using traditional solutions, individuals must be analyzed using each of a plurality of individualized prediction techniques. To this end, multi-label classification techniques in the healthcare industry, as well as other industries, consume vast amounts of processing and memory resources the requirements of which grow exponentially with the complexity of the multi-label classification domain.

The ultimate goal of a multi-label classification solution is to fully document conditions for each given member in a membership base. Referred to as documentation accuracy, a problem exists where members are not fully and/or accurately documented for all their respective conditions. Traditional techniques for addressing documentation inaccuracies may comprise employing deterministic and/or machine learning solutions to predict whether a codes is missing from a member's data records (e.g., automated coding). Another traditional technique may comprise imputing code for members who match signature patterns of one or more other members with a specific code and therefore is most likely undocumented. Because each code may require specific prediction techniques, full documentation accuracy is currently limited by the processing and memory capacities of computers.

Various embodiments of the present disclosure make important contributions to traditional multi-label classification techniques by addressing these technical challenges, among others.

### BRIEF SUMMARY

In general, various embodiments of the present disclosure provide methods, apparatus, systems, computing devices, computing entities, and/or the like improved multi-classification in complex prediction domains. Various embodiments of the present disclosure make important technical contributions to data prediction and imputation that address the efficiency and reliability shortcomings of existing multi-classification solutions by generating a singular correlated prediction based on a plurality of non-correlated predictions. As described herein, predictive data analysis computing systems may be configured to perform multi-label classification on data records to identify if one or more data elements, variables, or features are missing or should be imputed, rather than a single prediction representing a member likelihood of at least one data element, variable, or feature (from a plurality of data elements, variables, or features) that is missing/suspected. Accordingly, by transforming a plurality of non-correlated predictions into a correlated prediction based on simulation data realizations associated with the plurality of non-correlated predictions, the techniques described herein improve accuracy and speed of predictive operations as needed on downstream processing of data records for missing data, while reducing the computing resources needed to accomplish full documentation accuracy.

In some embodiments, a computer-implemented method comprises generating, by one or more processors, a correlation matrix based on a plurality of co-occurrence values associated with a plurality of reference non-correlated predictions; generating, by the one or more processors, a simulation matrix comprising a plurality of simulation data records based on a number of simulation instances and the plurality of reference non-correlated predictions; generating, by the one or more processors, a plurality of correlated simulation data records based on the correlation matrix and select ones of the plurality of simulation data records; generating, by the one or more processors, one or more univariates based on the plurality of correlated simulation data records; generating, by the one or more processors, a correlated prediction by comparing the one or more univariates with a plurality of input non-correlated predictions associated with an input data record; and identifying, by the one or more processors, at least one missing data feature from a plurality of data features associated with the plurality of reference non-correlated predictions that is missing from the input data record based on the correlated prediction.

In some embodiments, a computing system comprising memory and one or more processors communicatively coupled to the memory, the one or more processors configured to generate a correlation matrix based on a plurality of co-occurrence values associated with a plurality of reference non-correlated predictions; generate a simulation matrix comprising a plurality of simulation data records based on a number of simulation instances and the plurality of reference non-correlated predictions; generate a plurality of correlated simulation data records based on the correlation matrix and select ones of the plurality of simulation data records; generate one or more univariates based on the plurality of correlated simulation data records; generate a correlated prediction by comparing the one or more univariates with a plurality of input non-correlated predictions associated with an input data record; and identify at least one missing data feature from a plurality of data features associated with the plurality of reference non-correlated predictions that is missing from the input data record based on the correlated prediction.

In some embodiments, one or more non-transitory computer-readable storage media including instructions that, when executed by one or more processors, cause the one or more processors to generate a correlation matrix based on a plurality of co-occurrence values associated with a plurality of reference non-correlated predictions; generate a simulation matrix comprising a plurality of simulation data records based on a number of simulation instances and the plurality of reference non-correlated predictions; generate a plurality of correlated simulation data records based on the correlation matrix and select ones of the plurality of simulation data records; generate one or more univariates based on the plurality of correlated simulation data records; generate a correlated prediction by comparing the one or more univariates with a plurality of input non-correlated predictions associated with an input data record; and identify at least one missing data feature from a plurality of data features associated with the plurality of reference non-correlated predictions that is missing from the input data record based on the correlated prediction.

### BRIEF DESCRIPTION OF THE DRAWINGS

FIG. 1 provides an example overview of an architecture in accordance with some embodiments of the present disclosure.

FIG. 2 provides an example predictive data analysis computing entity in accordance with some embodiments of the present disclosure.

FIG. 3 provides an example client computing entity in accordance with some embodiments of the present disclosure.

FIG. 4 is a flowchart diagram of an example process for analyzing data records in accordance with some embodiments of the present disclosure.

FIG. 5 is a flowchart diagram of an example process for generating correlated predictions in accordance with some embodiments of the present disclosure.

FIG. 6 is an operation example of an example correlation matrix in accordance with some embodiments of the present disclosure.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

### I. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

A non-volatile computer-readable storage medium may include a floppy disk, flexible disk, hard disk, solid-state storage (SSS) (e.g., a solid-state drive (SSD), solid-state card (SSC), solid-state module (SSM)), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

A volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises a combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specifically-configured machines performing the steps or operations specified in the block diagrams and flowchart illustrations. Accordingly, the block diagrams and flowchart illustrations support various combinations of embodiments for performing the specified instructions, operations, or steps.

### II. Example Framework

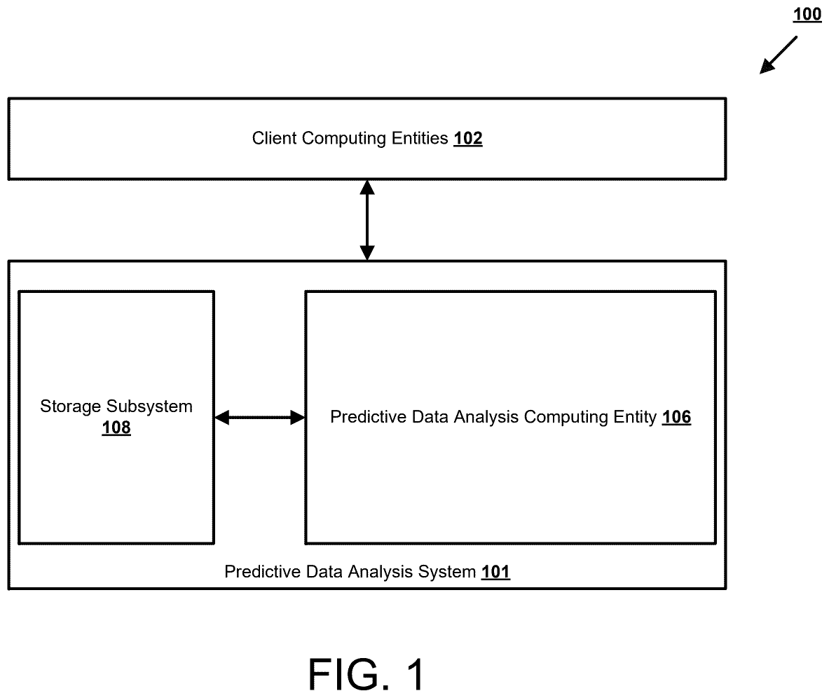

FIG. 1 provides an example overview of an architecture 100 in accordance with some embodiments of the present disclosure. The architecture 100 includes a predictive data analysis system 101 configured to receive predictive data analysis requests from client computing entities 102, process the predictive data analysis requests to generate predictions, provide the generated predictions to the client computing entities 102, and automatically initiate performance of prediction-based actions based on the generated predictions. The example architecture 100 may be used in a plurality of domains and not limited to any specific application as disclosed herewith. The plurality of domains may include banking, healthcare, industrial, manufacturing, education, retail, to name a few.

An example of a prediction-based action that may be performed using the predictive data analysis system 101 comprises receiving a request for predicting missing classification data (e.g., hierarchical classification), such as missing co-morbidities from an EHR of a patient, predicting missing classification data from the EHR, and displaying the predicted missing classification data on a user interface. Other examples of prediction-based actions comprise generating a diagnostic report, displaying/providing resources, generating, and/or executing action scripts, generating alerts or reminders, or generating one or more electronic communications based on the predicted missing classification data.

In accordance with various embodiments of the present disclosure, a plurality of non-correlated predictions, associated with data features that are present in a plurality of data records, is transformed into a correlated prediction based on simulation data realizations associated with the plurality of non-correlated predictions. The simulation data may comprise co-occurrence information that is applied to the plurality of non-correlated predictions to generate correlated data. The correlated data may be compared with an input data record to generate a correlated prediction comprising a probability that at least one of the data features present in the plurality of data records is missing from or should be present in the input data record. In this manner, some of the techniques of the present disclosure, improve accuracy and speed of predictive operations as needed on downstream processing of data records for missing data. Accordingly, the techniques described herein improve the computational efficiency, storage-wise efficiency, and/or speed of training predictive machine learning models.

In some embodiments, predictive data analysis system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software, and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more client computing entities 102, process the predictive data analysis requests to generate predictions corresponding to the predictive data analysis requests, provide the generated predictions to the client computing entities 102, and automatically initiate performance of prediction-based actions based on the generated predictions.

The storage subsystem 108 may be configured to store input data used by the predictive data analysis computing entity 106 to perform predictive data analysis as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### A. Example Predictive Data Analysis Computing Entity

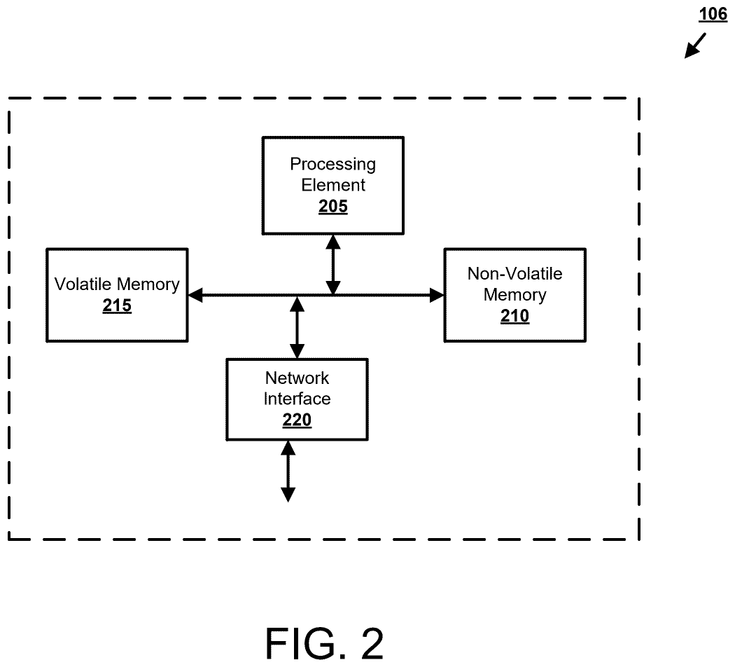

FIG. 2 provides an example predictive data analysis computing entity 106 in accordance with some embodiments of the present disclosure. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In some embodiments, these functions, operations, and/or processes may be performed on data, content, information, and/or similar terms used herein interchangeably.

As shown in FIG. 2, in some embodiments, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In some embodiments, the predictive data analysis computing entity 106 may further include, or be in communication with, non-volatile media (also referred to as non-volatile storage, memory, memory storage, memory circuitry, and/or similar terms used herein interchangeably). In some embodiments, the non-volatile storage or memory may include one or more non-volatile memory 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In some embodiments, the predictive data analysis computing entity 106 may further include, or be in communication with, volatile media (also referred to as volatile storage, memory, memory storage, memory circuitry, and/or similar terms used herein interchangeably). In some embodiments, the volatile storage or memory may also include one or more volatile memory 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in some embodiments, the predictive data analysis computing entity 106 may also include one or more network interfaces 220 for communicating with various computing entities, such as by communicating data, content, information, and/or similar terms used herein interchangeably that may be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### B. Example Client Computing Entity

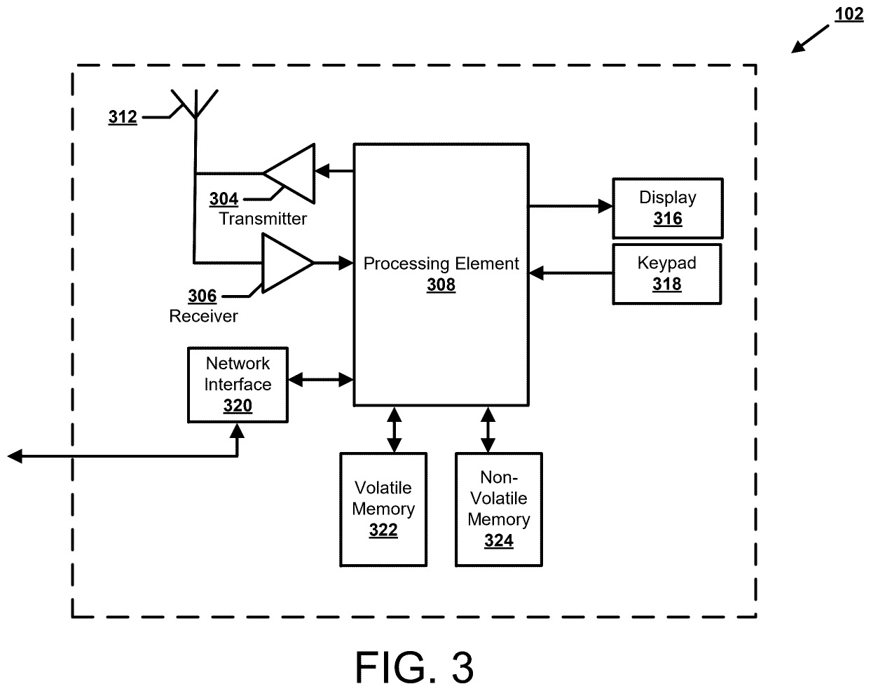

FIG. 3 provides an example client computing entity in accordance with some embodiments of the present disclosure. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 may be operated by various parties. As shown in FIG. 3, the client computing entity 102 may include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In some embodiments, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 may communicate with various other entities using mechanisms such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 may also download changes, add-ons, and updates, for instance, to its firmware, software (e.g., including executable instructions, applications, program modules), and operating system.

According to some embodiments, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In some embodiments, the location module may acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data may be collected using a variety of coordinate systems, such as the DecimalDegrees (DD); Degrees, Minutes, Seconds (DMS); Universal Transverse Mercator (UTM); Universal Polar Stereographic (UPS) coordinate systems; and/or the like. Alternatively, the location information/data may be determined by triangulating the position of the client computing entity 102 in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops), and/or the like. For instance, such technologies may include the iBeacons, Gimbal proximity beacons, Bluetooth Low Energy (BLE) transmitters, NFC transmitters, and/or the like. These indoor positioning aspects may be used in a variety of settings to determine the location of someone or something to within inches or centimeters.

The client computing entity 102 may also comprise a user interface (that may include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the predictive data analysis computing entity 106, as described herein. The user input interface may comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 may include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface may be used, for example, to activate or deactivate certain functions, such as screen savers and/or sleep modes.

The client computing entity 102 may also include volatile memory 322 and/or non-volatile memory 324, which may be embedded and/or may be removable. For example, the non-volatile memory 324 may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory 322 may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile memory may store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the client computing entity 102 or accessible through a browser or other user interface for communicating with the predictive data analysis computing entity 106 and/or various other computing entities.

In another embodiment, the client computing entity 102 may include one or more components or functionalities that are the same or similar to those of the predictive data analysis computing entity 106, as described in greater detail above. As will be recognized, these architectures and descriptions are provided for example purposes only and are not limited to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### III. Examples of Certain Terms

In some embodiments, the term “prediction” refers to a data construct that describes an output generated by a predictive machine learning model based on input data. A prediction may comprise a value generated by a predictive machine learning for a data feature or variable value (e.g., of a given data record) that is unknown, and of which, a prediction for the data feature or variable value is desired. A predictive machine learning model may generate a prediction based on training data used to train the predictive machine learning model. For example, a prediction may be generated by a predictive machine learning model based on an observation of example data features and/or variable values from training data. According to various embodiments of the present disclosure, training data comprising a plurality of training data records that is labeled with one or more data features or variable values may be used to train a predictive machine learning model to generate one or more predictions for one or more data records. In some embodiments, the one or more predictions comprise one or more values (e.g., probabilities) associated with a probable assignment of the one or more data features or variable values to the one or more data records. In some embodiments, a prediction comprises a score representative of a probability that a hierarchical classification associated with a disease or condition is missing or should be present in a data record.

In some embodiments, the term “hierarchical classification” refers to a data construct that describes a grouping of articles, conditions, objects, events, or subjects. A hierarchical classification may be assigned to a data record as a data feature or variable value associated with the data record with respect to a plurality of classification tiers. In some example embodiments, a hierarchical classification comprises a disease/condition category and one or more International Classification of Diseases (ICD) codes associated with the disease/condition category. A given hierarchical classification may be dependent or independent from other hierarchical classifications. In some embodiments, a hierarchical classification comprises one or more positive or negative correlations to one or more other hierarchical classifications.

In some embodiments, the term “data record” refers to a data construct that describes an entry in a database comprising one or more data fields. A data record may be used to store, capture, or document one or more data features and/or variables associated with a subject. For example, one or more values may be stored to one or more data fields (of a data record) associated with one or more data features and/or variables. In one example embodiment, a data record comprises an electronic health/medical record comprising one or more data fields associated with one or more diagnostic codes (e.g., ICD) or one or more hierarchical classifications associated with the one or more diagnostic codes usable to document diseases or conditions of a patient.

In some embodiments, the term “non-correlated prediction” refers to a prediction associated with a data feature or variable value that is independent of another prediction associated with another data feature or variable value. A non-correlated prediction may be generated for a data record with respect to a given data feature or variable value, such as a particular hierarchical classification. In some embodiments, a plurality of non-correlated predictions is generated for a data record, wherein generating the plurality of non-correlated predictions comprises generating a prediction for each of a plurality of data features or variable values. In some example embodiments, the plurality of non-correlated predictions comprises a plurality of probabilities that the data record is associated with (or missing) a respective plurality of hierarchical classifications, where each of the plurality of non-correlated predictions is associated with a particular one of the plurality of hierarchical classifications.

In some embodiments, the term “correlation matrix” refers to a data construct that describes a plurality of values representative of a plurality of correlations between a plurality of data features or variables, such as hierarchical classifications. According to various embodiments of the present disclosure, a correlation matrix is generated based on a plurality of co-occurrence values associated with a plurality of reference non-correlated predictions associated with a plurality of hierarchical classifications assigned to a plurality of reference data records. A correlation matrix associated with correlations between a plurality of hierarchical classifications may be generated based on empirical data as well as predicted data. For example, co-occurrence values between hierarchical classifications may be calculated based on actual assignments of a plurality of hierarchical classifications to given ones of a plurality of reference data records as well as predictions (e.g., generated by using a predictive machine learning model) of probable (e.g., that may be missing) hierarchical classification assignments to the plurality of reference data records.

In some embodiments, the term “co-occurrence value” refers to a data construct that describes a measure of tendency for two or more data features or variable values occurring together. According to various embodiments of the present disclosure, a plurality of co-occurrence values may be representative of degrees of correlation between a plurality of reference non-correlated predictions associated with a plurality of hierarchical classifications. For example, a co-occurrence value associated with two hierarchical classification predictions may be representative of a frequency in which two hierarchical classifications are both predicted to be assigned to a given data record. In some embodiments, co-occurrence values may be in a range between ‘−1’ to ‘1,’ where a value of ‘1’ may represent a perfect correlation, a value of ‘0’ may represent no correlation, and a value of ‘−1’ may represent an anti-correlation. In some embodiments, a co-occurrence value may be determined based on a Pearson correlation coefficient. According to various embodiments of the present disclosure, a correlation matrix is generated based on a plurality of co-occurrence values associated with a plurality of reference non-correlated predictions for a plurality of hierarchical classifications.

In some embodiments, the term “simulation data record” refers to a data construct that describes a data record generated via a simulation, such as a Monte Carlo simulation. For example, a simulation may generate a simulation dataset comprising a plurality of simulation data records. Members (e.g., simulation data records) of a simulation dataset may comprise values associated with data features and/or variables that are determined based on a given probability distribution associated with the simulation dataset. For example, a probability distribution may define a probability of occurrence of different possible data features and/or variable values for a plurality of simulation data records belonging to a simulation dataset. According to various embodiments of the present disclosure, a simulation data record comprises one or more data features and/or variable values that are selected based on a random normal distribution.

In some embodiments, the term “simulation” refers to a generation of one or more simulation data records associated with outcomes selected from a plurality of possible outcomes. For example, a simulation may generate a plurality of simulation data records comprising data features and/or variable values that are selected based on a probability distribution, such as a random normal distribution. In some embodiments, a simulation comprises repeated selection from possible data inputs based on a probability distribution of the possible data inputs, e.g., a Monte Carlo simulation. According to various embodiments of the present disclosure, a simulation may comprise selectively assigning a plurality of hierarchal classifications to a plurality of simulation data records based on a given probability distribution.

In some embodiments, the term “simulation matrix” refers to a data construct that describes a simulation dataset comprising a plurality of simulation data records, associated with a plurality of simulation instances, comprising data features and/or variable values that are (i) associated with a plurality of reference non-correlated predictions and (ii) selected based on a probability distribution, such as a random normal distribution. For example, a simulation matrix may comprise simulation data records generated based on a Monte Carlo simulation. A simulation matrix may comprise a size of M×N where M may represent a number of simulation instances and N may represent a number of possible data features and/or variable values (e.g., associated with a plurality of reference non-correlated predictions). According to various embodiments of the present disclosure, a simulation matrix comprises (i) a first dimension associated with a number of simulation instances performed and (ii) a second dimension associated with one or more possible data features or variable values (e.g., hierarchical classifications that may be used to classify a simulation data record) for each simulation instance. The number of simulation instances may be configurable to any value and may be varied based on, for example, a desired accuracy and/or speed.

In some embodiments, the term “correlated simulation data record” refers to a simulation data record selected from a simulation matrix and correlated with one or more other simulation data records also selected from the simulation matrix based on a correlation matrix. According to various embodiments of the present disclosure, a plurality of correlated simulation data records, comprising a correlated simulation matrix, is generated by (i) generating a Cholesky decomposition matrix based on a correlation matrix and (i) applying a dot product of the Cholesky decomposition matrix to select ones of a plurality of simulation data records selected from a simulation matrix.

In some embodiments, the term “univariate” refers to a data construct that describes a function associated with a single data feature or variable of a data record. For example, a univariate may comprise a value representative of a probability of correlation of a data feature or variable value, such as a hierarchical classification, associated with a correlated simulation data record with another data feature or variable value (e.g., hierarchical classification) based on a degree of correlation between the data feature or variable value with another data feature or variable value. According to various embodiments of the present disclosure, one or more univariates are generated based on a normal cumulative distribution function of a correlated simulation matrix comprising a plurality of correlated simulation data records. A normal cumulative distribution function may comprise a probability of a data feature or variable comprising a value less than or equal to a specific value. For example, a normal cumulative distribution function may be applied to a correlated simulation matrix to determine a probability of correlation (e.g., in the range of [0.0, 1.0]) of data features and/or variable values, such as hierarchical classifications, based on correlated data features and/or variable values of correlated simulation data records associated with the correlated simulation matrix.

In some embodiments, the term “correlated prediction” refers to a data construct that describes a prediction associated with a feature or variable value that is based on a plurality of non-correlated predictions. A correlated prediction may be generated based on one or more correlations, obtained via simulation data, of one or more data features and/or variable values associated with a plurality of non-correlated predictions. For example, a correlated prediction may be generated by comparing a plurality of input non-correlated predictions, generated by an upstream predictive machine learning model, with one or more univariates associated with correlated simulation values. A correlated prediction may be representative of a projection (or realization) of global correlation outcomes (e.g., of one or more simulated data features and/or variable values) associated with a plurality of reference data records onto one or more independent predictions of one or more data features and/or variable values for a given input data object. According to various embodiments of the present disclosure, a correlated prediction comprises a dependent probability of at least one hierarchical classification being present (or absent) based on an input of a plurality of independent hierarchical classification probabilities (e.g., input non-correlated predictions).

In some embodiments, the term “hierarchical relationship” refers to a data construct that describes a relative categorization comprising a generalization or refinement within a plurality of classification tiers. For example, a hierarchical classification may comprise a hierarchical relationship with another hierarchical classification that may be above or below representative of a level of classification broadening or narrowing, respectively. In some embodiments, a hierarchical relationship comprises a parent-child relationship within a tree structure. A child node in a tree structure may comprise a subset of a parent node. That is, a parent node (e.g., classification) may be refined by one or more child nodes connected to the parent node. In some embodiments, (i) a first hierarchical classification, associated with a first prediction of the plurality of input non-correlated predictions, is determined as assigned to an input data record, and (ii) the first prediction and a second prediction of the plurality of input non-correlated predictions is modified based on (a) the determination of the first hierarchical classification and (b) a hierarchical relationship between the first hierarchical classification and a second hierarchical classification associated with the second prediction.

In some embodiments, the term “predictive machine learning model” refers to a data construct that describes parameters, hyperparameters, and/or defined operations of a machine learning model that is configured to generate one or more predictions associated with one or more input data records. In some embodiments, the predictive machine learning model is configured to or used to generate a plurality of reference non-correlated predictions associated with a plurality of data features or variable values, where a correlation matrix is generated based on a plurality of co-occurrence values associated with the plurality of reference non-correlated predictions.

### IV. Overview

Various embodiments of the present disclosure make important technical contributions to data prediction and imputation that address the efficiency and reliability shortcomings of existing multi-label classification solutions. For example, some techniques of the present disclosure improve the predictive accuracy of a predictive machine learning model used in predicting missing data from data records. To do so, prediction outputs associated with multi-label classification performed by the predictive machine learning model may be correlated and used to generate a singular value representative of a plurality of individual predictions. By doing so, some of the techniques of the present disclosure may enhance the predictive outputs of the predictive machine learning model by transforming a plurality of non-correlated predictions into one or more correlated predictions. This, in turn, leads to a reduction in the computing resources, such as memory and processing power, required to facilitate multi-classification solutions.

Various embodiments of the present disclosure improve the predictive accuracy of predictive machine learning models by generating a singular correlated prediction based on a plurality of non-correlated predictions. As described herein, it may be desirable to detect missing information in data records. Predictive data analysis computing systems may be configured to impute missing data in data records by matching characteristics or patterns of similar data records. As an example, predictive data analysis computing systems may be configured to perform multi-label classification on data records to identify if one or more data elements, variables, or features are missing or should be imputed. Multi-label classification may comprise generating an individual score for each data element, variable, or feature representative of a pseudo-probability of a likelihood that a data record is missing (or should be present) the data element, variable, or feature. In certain circumstances, a single prediction representing a member likelihood of at least one data element, variable, or feature (from a plurality of data elements, variables, or features) that is missing/suspected may be desired. However, merely combining a plurality of individual probabilities into a single probability would create an assumption that each prediction is statistically independent when in fact they are typically correlated.

In accordance with various embodiments of the present disclosure, a plurality of non-correlated predictions, associated with data features that are present in a plurality of data records, is integrated into a correlated prediction based on simulation data realizations associated with the plurality of non-correlated predictions. The simulation data may comprise co-occurrence information that is applied to the plurality of non-correlated predictions to generate correlated data. The correlated data may be compared with an input data record to generate a correlated prediction comprising a probability that at least one of the data features present in the plurality of data records is missing from or should be present in the input data record. In this manner, some of the techniques of the present disclosure, improve accuracy and speed of predictive operations as needed on downstream processing of data records for missing data.

In accordance with various embodiments of the present disclosure, correlated predictions may be generated to identify missing data in data records by correlating a plurality of non-correlated predictions based on simulation data. By doing so, incomplete data records may be intelligently identified based on a single prediction (score) rather than the more memory intensive pluralities of individual predictions. In this way, some of the techniques of the present disclosure may be practically applied, in real time, to improve and prioritize data correction workflows, while reducing the computing resources traditionally expended to do so.

Moreover, some of the techniques (e.g., prediction correlation techniques, etc.) of the present disclosure reduces the number of computational operations needed and/or the amount of training data entries needed to train predictive machine learning models. Accordingly, the techniques described herein improve the computational efficiency, storage-wise efficiency, and/or speed of training predictive machine learning models. Other technical improvements and advantages may be realized by one of ordinary skill in the art.

Examples of technologically advantageous embodiments of the present disclosure include: (i) prediction fusion techniques of fusing insights from traditionally disparate prediction into an encompassing dependent prediction, (ii) real-time prediction techniques for improving data record quality, (iii) machine learning training techniques for improving utilization of predictive machine learning model outputs while reducing computational resource usage, among others. Other technical improvements and advantages may be realized by one of ordinary skill in the art.

### V. Example System Operations

As indicated, various embodiments of the present disclosure make important technical contributions to data prediction and imputation that address the efficiency and reliability shortcomings of existing multi-label classification solutions by generating a singular correlated prediction based on a plurality of non-correlated predictions. By doing so, incomplete data records may be intelligently identified based on a single prediction (score). In this way, some of the techniques of the present disclosure may be practically applied, in real time, to improve and prioritize data correction workflows, while reducing the computing resources required to do so.

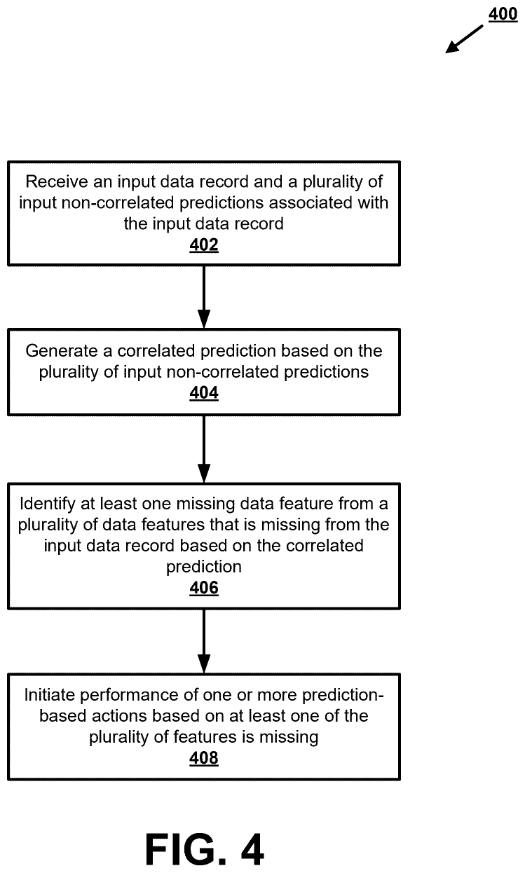

FIG. 4 is a flowchart diagram of an example process for analyzing data records in accordance with some embodiments of the present disclosure.

In some embodiments, via the various steps/operations of the process 400, the predictive data analysis computing entity 106 may generate a correlated prediction for an input data record and use the correlated prediction to identify at least one missing data feature from a plurality of data features that is missing from or should be present in the input data record.

In some embodiments, the process 400 begins at step/operation 402 when the predictive data analysis computing entity 106 receives an input data record and a plurality of input non-correlated predictions associated with the input data record. In some example embodiments, the input data record may be received by the predictive data analysis computing entity 106 as a request for analyzing whether the input data record is missing data. The request may comprise either a singular request or as part of a batch request for analyzing a plurality of input data records.

In some embodiments, a data record describes an entry in a database comprising one or more data fields. A data record may be used to store, capture, or document one or more data features and/or variables associated with a subject. For example, one or more values may be stored to one or more data fields (of a data record) associated with one or more data features and/or variables. In one example embodiment, a data record comprises an electronic health/medical record comprising one or more data fields associated with one or more diagnostic codes (e.g., ICD) or one or more hierarchical classifications associated with the one or more diagnostic codes usable to document diseases or conditions of a patient.

In some embodiments, the plurality of input non-correlated predictions associated with the input data record may be received as part of the request for analyzing whether the input data record is missing data or received as part of a workflow for processing the request by the predictive data analysis computing entity 106. In some embodiments, the plurality of input non-correlated predictions may be associated with independent predictions on a plurality of data features or variable values for the input data record.

In some embodiments, a prediction describes an output generated by a predictive machine learning model based on input data, such as an input data record. A prediction may comprise a value generated by a predictive machine learning for a data feature or variable value (e.g., of a given data record) that is unknown, and of which, a prediction for the data feature or variable value is desired. In some embodiments, a predictive machine learning model describes parameters, hyperparameters, and/or defined operations of a machine learning model that is configured to generate one or more predictions associated with one or more input data records.

A predictive machine learning model may generate a prediction based on training data used to train the predictive machine learning model. For example, a prediction may be generated by a predictive machine learning model based on an observation of example data features and/or variable values from training data. According to various embodiments of the present disclosure, training data comprising a plurality of training data records that is labeled with one or more data features or variable values may be used to train a predictive machine learning model to generate one or more predictions for one or more data records. In some embodiments, the one or more predictions comprise one or more values (e.g., probabilities) associated with a probable assignment of the one or more data features or variable values to the one or more data records. In some embodiments, a prediction comprises a score representative of a probability that a hierarchical classification associated with a disease or condition is missing or should be present in a data record.

In some embodiments, a non-correlated prediction comprises a prediction associated with a data feature or variable value that is independent of another prediction associated with another data feature or variable value. A non-correlated prediction may be generated for a data record with respect to a given data feature or variable value, such as a particular hierarchical classification. In some embodiments, a plurality of non-correlated predictions is generated for a data record, wherein generating the plurality of non-correlated predictions comprises generating a prediction for each of a plurality of data features or variable values. In some example embodiments, the plurality of non-correlated predictions comprises a plurality of probabilities that the data record is associated with (or missing) a respective plurality of hierarchical classifications, where each of the plurality of non-correlated predictions is associated with a particular one of the plurality of hierarchical classifications.

In some embodiments, a hierarchical classification describes a grouping of articles, conditions, objects, events, or subjects. A hierarchical classification may be assigned to a data record as a data feature or variable value associated with the data record with respect to a plurality of classification tiers. In some example embodiments, a hierarchical classification comprises a disease/condition category and one or more ICD codes associated with the disease/condition category. A given hierarchical classification may be dependent or independent from other hierarchical classifications. In some embodiments, a hierarchical classification comprises one or more positive or negative correlations to one or more other hierarchical classifications.

A hierarchical classification may comprise a hierarchical relationship with another hierarchical classification that may be above or below representative of a level of classification broadening or narrowing, respectively. In some embodiments, a hierarchical relationship describes a relative categorization comprising a generalization or refinement within a plurality of classification tiers. In some embodiments, a hierarchical relationship comprises a parent-child relationship within a tree structure. A child node in a tree structure may comprise a subset of a parent node. That is, a parent node (e.g., classification) may be refined by one or more child nodes connected to the parent node. In some embodiments, (i) a first hierarchical classification, associated with a first prediction of the plurality of input non-correlated predictions, is determined as assigned to an input data record, and (ii) the first prediction and a second prediction of the plurality of input non-correlated predictions is modified based on (a) the determination of the first hierarchical classification and (b) a hierarchical relationship between the first hierarchical classification and a second hierarchical classification associated with the second prediction.

In some embodiments, at step/operation 404, the predictive data analysis computing entity 106 generates a correlated prediction based on the plurality of input non-correlated predictions.

In some embodiments, a correlated prediction describes a prediction associated with a feature or variable value that is based on a plurality of non-correlated predictions. A correlated prediction may be generated based on one or more correlations, obtained via simulation data, of one or more data features and/or variable values associated with a plurality of non-correlated predictions. A correlated prediction may be representative of a projection (or realization) of global correlation outcomes (e.g., of one or more simulated data features and/or variable values) associated with a plurality of reference data records onto one or more independent predictions of one or more data features and/or variable values for a given input data object. According to some example embodiments, a correlated prediction comprises a dependent probability of at least one hierarchical classification being present (or absent) based on an input of a plurality of independent hierarchical classification probabilities (e.g., input non-correlated predictions).

According to various embodiments of the present disclosure, a correlated prediction may be generated by comparing a plurality of input non-correlated predictions associated with an input data record with one or more univariates associated with correlated simulation values.

In some embodiments, a correlated prediction may be generated based on the following:

$$P = \frac{{\sum }_{k} ( ( {\sum }_{n} {u}_{j} < {p}_{i} ) < 0 )}{k} \qquad Equation 1$$

where P may represent a probability of at least one feature or variable value of a plurality of features or variable values being present, pᵢ may represent each input non-correlated prediction compared to one or more univariates uⱼ, k may represent a number of simulations, and n may represent a number of the plurality of features or variables.

In some embodiments, a univariate describes a function associated with a single data feature or variable of a data record. For example, a univariate may comprise a value representative of a probability of correlation of a data feature or variable value, such as a hierarchical classification, associated with a correlated simulation data record with another data feature or variable value (e.g., hierarchical classification) based on a degree of correlation between the data feature or variable value with another data feature or variable value.

In some embodiments, at step/operation 406, the predictive data analysis computing entity 106 identifies at least one missing data feature from a plurality of data features that is missing from the input data record based on the correlated prediction. In some embodiments, the plurality of data features is associated with a plurality of reference non-correlated predictions of a plurality of reference data records. A simulation matrix used to generate the correlated prediction may be generated based on the reference non-correlated predictions.

In some embodiments, at step/operation 408, the predictive data analysis computing entity 106 initiates performance of one or more prediction-based actions based on at least one of the plurality of data features is missing. Initiating the performance of the one or more prediction-based actions based on at least one of the plurality of data features is missing comprises, for example, performing a resource-based action (e.g., allocation of resource), generating a diagnostic report, displaying/providing resources, generating, and/or executing action scripts, generating alerts or reminders, or generating one or more electronic communications. The one or more prediction-based actions may further include displaying visual renderings of the aforementioned examples of prediction-based actions in addition to values, charts, and representations associated with the correlated prediction and/or missing data feature(s) using a prediction output user interface.

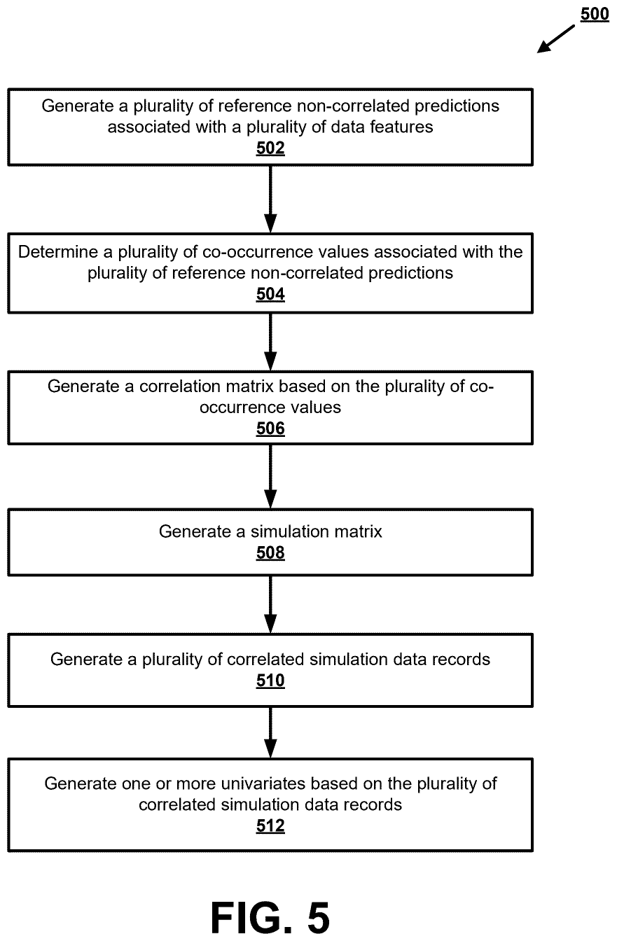

FIG. 5 is a flowchart diagram of an example process for generating correlated predictions in accordance with some embodiments of the present disclosure.

In some embodiments, the process 500 begins at step/operation 502 when the predictive data analysis computing entity 106 generates, using a predictive machine learning model, a plurality of reference non-correlated predictions associated with a plurality of data features and/or variable values. The plurality of reference non-correlated predictions may be generated for a plurality of reference data records. In some embodiments, an input data record may be analyzed for missing data based on the plurality of reference non-correlated predictions as further discussed herein.

In some embodiments, at step/operation 504, the predictive data analysis computing entity 106 determines a plurality of co-occurrence values associated with the plurality of reference non-correlated predictions. For example, for each of the plurality of reference data records, a co-occurrence value may be generated for pairs of the plurality of reference non-correlated predictions.

In some embodiments, a co-occurrence value describes a measure of tendency for two or more data features or variable values occurring together. According to various embodiments of the present disclosure, a plurality of co-occurrence values may be representative of degrees of correlation between a plurality of reference non-correlated predictions associated with a plurality of hierarchical classifications. For example, a co-occurrence value associated with two hierarchical classification predictions may be representative of a frequency in which two hierarchical classifications are both predicted to be assigned to a given data record. In some embodiments, co-occurrence values may be in a range between ‘−1’ to ‘1,’ where a value of ‘1’ may represent a perfect correlation, a value of ‘0’ may represent no correlation, and a value of ‘−1’ may represent an anti-correlation. In some embodiments, a co-occurrence value may be determined based on a Pearson correlation coefficient. In some other embodiments, a co-occurrence value may be determined based on other correlation measures.

In some embodiments, at step/operation 506, the predictive data analysis computing entity 106 generates a correlation matrix based on the plurality of co-occurrence values.

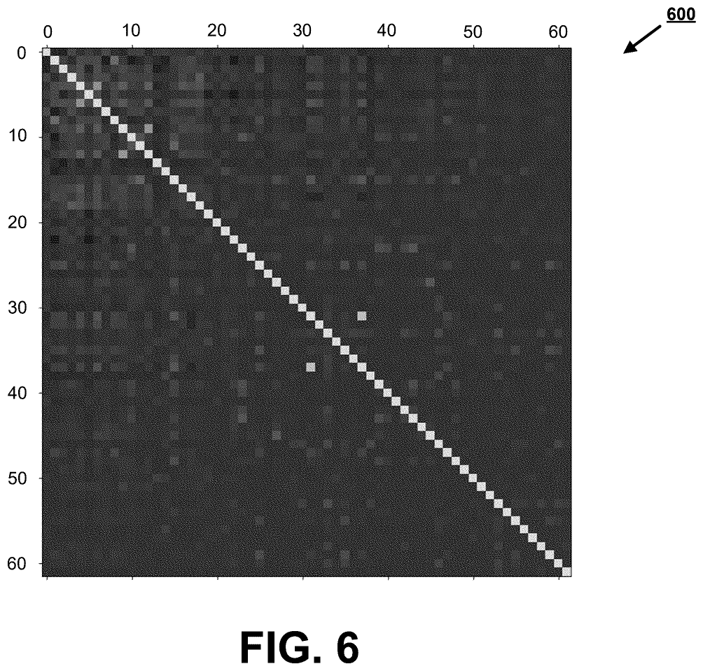

FIG. 6 depicts an operation example of a correlation matrix 600. The correlation matrix 600 describes a plurality of values representative of a plurality of correlations between a plurality of data features or variables, such as hierarchical classifications. According to various embodiments of the present disclosure, the correlation matrix 600 is generated based on a plurality of co-occurrence values associated with a plurality of reference non-correlated predictions associated with a plurality of hierarchical classifications assigned to a plurality of reference data records. A correlation matrix 600 associated with correlations between a plurality of hierarchical classifications may be generated based on empirical data as well as predicted data. For example, co-occurrence values between hierarchical classifications may be calculated based on actual assignments of a plurality of hierarchical classifications to given ones of a plurality of reference data records as well as predictions (e.g., generated by using a predictive machine learning model) of probable (e.g., that may be missing) hierarchical classification assignments to the plurality of reference data records.

Returning to FIG. 5, in some embodiments, at step/operation 508, the predictive data analysis computing entity 106 generates a simulation matrix comprising a plurality of simulation data records based on a number of simulation instances and the plurality of reference non-correlated predictions.

In some embodiments, a simulation matrix describes a simulation dataset comprising a plurality of simulation data records, associated with a plurality of simulation instances, comprising data features and/or variable values that are (i) associated with a plurality of reference non-correlated predictions and (ii) selected based on a probability distribution, such as a random normal distribution. For example, a simulation matrix may comprise simulation data records generated based on a Monte Carlo simulation. A simulation matrix may comprise a size of M×N where M may represent a number of simulation instances and N may represent a number of possible data features and/or variable values (e.g., associated with a plurality of reference non-correlated predictions). According to various embodiments of the present disclosure, a simulation matrix comprises (i) a first dimension associated with a number of simulation instances performed and (ii) a second dimension associated with one or more possible data features or variable values (e.g., hierarchical classifications that may be used to classify a simulation data record) for each simulation instance. The number of simulation instances may be configurable to any value and may be varied based on, for example, a desired accuracy and/or speed.

In some embodiments, a simulation data record describes a data record generated via a simulation, such as a Monte Carlo simulation. For example, a simulation may generate a simulation dataset comprising a plurality of simulation data records. Members (e.g., simulation data records) of a simulation dataset may comprise values associated with data features and/or variables that are determined based on a given probability distribution associated with the simulation dataset. For example, a probability distribution may define a probability of occurrence of different possible data features and/or variable values for a plurality of simulation data records belonging to a simulation dataset. According to various embodiments of the present disclosure, a simulation data record comprises one or more data features and/or variable values that are selected based on a random normal distribution.

In some embodiments, a simulation describes a generation of one or more simulation data records associated with outcomes selected from a plurality of possible outcomes. For example, a simulation may generate a plurality of simulation data records comprising data features and/or variable values that are selected based on a probability distribution, such as a random normal distribution. In some embodiments, a simulation comprises repeated selection from possible data inputs based on a probability distribution of the possible data inputs, e.g., a Monte Carlo simulation. According to various embodiments of the present disclosure, a simulation may comprise selectively assigning a plurality of hierarchal classifications to a plurality of simulation data records based on a given probability distribution.

In some embodiments, at step/operation 510, the predictive data analysis computing entity 106 generates a plurality of correlated simulation data records based on the correlation matrix and select ones of the plurality of simulation data records. Generating the plurality of correlated simulation data records may comprise sampling simulated data records from a random normal distribution by selecting simulation data records from the simulation matrix and correlating the selected simulation data records with co-occurrence values associated with the correlation matrix.

In some embodiments, a correlated simulation data record describes a simulation data record selected from a simulation matrix and correlated with one or more other simulation data records, also selected from the simulation matrix, based on a correlation matrix. According to various embodiments of the present disclosure, generating a plurality of correlated simulation data records comprises generating a correlated simulation matrix by (i) generating a Cholesky decomposition matrix based on a correlation matrix and (i) applying a dot product of the Cholesky decomposition matrix to select ones of a plurality of simulation data records selected from a simulation matrix.

In some embodiments, a plurality of correlated simulation data records is determined by:

$${R}^{C} = R \cdot L \qquad Equation 2$$

where R^{C} may represent plurality of correlated simulation data records, R may represent samples (e.g., one or more simulation data records selected from a random normal distribution), and L may represent a Cholesky decomposition lower triangular matrix.

A Cholesky decomposition lower triangular matrix may be determined by:

$$C = {LL}^{T} \qquad Equation 3$$

where C may represent a correlation matrix, where L may represent the Cholesky decomposition lower triangular matrix and L^{T} may represent a transpose of the Cholesky decomposition lower triangular matrix.

In some embodiments, at step/operation 512, the predictive data analysis computing entity 106 generates one or more univariates based on the plurality of correlated simulation data records. According to various embodiments of the present disclosure, one or more univariates are generated based on a normal cumulative distribution function of a correlated simulation matrix comprising the plurality of correlated simulation data records. A normal cumulative distribution function may comprise a probability of a data feature or variable comprising a value less than or equal to a specific value. For example, a normal cumulative distribution function may be applied to a correlated simulation matrix to determine a probability of correlation (e.g., in the range of [0.0, 1.0]) of data features and/or variable values, such as hierarchical classifications, based on correlated data features and/or variable values of correlated simulation data records associated with the correlated simulation matrix.

Accordingly, as described above, various embodiments of the present disclosure make important technical contributions to predictive data analysis that address the efficiency and reliability shortcomings of existing predictive data analysis solutions. For example, some techniques of the present disclosure improve the predictive accuracy of a predictive machine learning model used in predicting missing data from data records. To do so, prediction outputs associated with multi-label classification performed by the predictive machine learning model may be simulated and interrelated into a singular value representative of a plurality of individual predictions. By doing so, some of the techniques of the present disclosure may enhance the predictive outputs of the predictive machine learning model by transforming a plurality of non-correlated predictions into one or more correlated predictions.

Some techniques of the present disclosure enable the generation of correlated predictions associated with missing data that may be used to initiate one or more predictive actions to achieve real-world effects. The data analysis techniques of the present disclosure may be used, applied, and/or otherwise leveraged to predict missing data from data records, which may help in data imputation. The missing data analysis of the present disclosure may be leveraged to initiate the performance of various computing tasks that improve the performance of a computing system (e.g., a computer itself, etc.) with respect to various predictive actions performed by the predictive data analysis computing entity 106. Example predictive actions may include performing a resource-based action (e.g., allocation of resource), generating a diagnostic report, displaying/providing resources, generating, and/or executing action scripts, generating alerts or reminders, or generating one or more electronic communications.

In some examples, the computing tasks may include predictive actions that may be based on a prediction domain. A prediction domain may include any environment in which computing systems may be applied to achieve real-word insights, such as predictions (e.g., of missing data), and initiate the performance of computing tasks, such as predictive actions e.g., updating user preferences or information, cancelling an account, adding an account, etc.) to act on the real-world insights. These predictive actions may cause real-world changes, for example, by controlling a hardware component, providing alerts, interactive actions, and/or the like.

Examples of prediction domains may include financial systems, clinical systems, autonomous systems, robotic systems, and/or the like. Predictive actions in such domains may include the initiation of automated instructions across and between devices, automated notifications, automated scheduling operations, automated precautionary actions, automated security actions, automated data processing actions, automated data compliance actions, automated data access enforcement actions, automated adjustments to computing and/or human data access management, and/or the like.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. Examples

Example 1. A computer-implemented method comprising: generating, by the one or more processors, a correlation matrix based on a plurality of co-occurrence values associated with a plurality of reference non-correlated predictions; generating, by the one or more processors, a simulation matrix comprising a plurality of simulation data records based on a number of simulation instances and the plurality of reference non-correlated predictions; generating, by the one or more processors, a plurality of correlated simulation data records based on the correlation matrix and select ones of the plurality of simulation data records; generating, by the one or more processors, one or more univariates based on the plurality of correlated simulation data records; generating, by the one or more processors, a correlated prediction by comparing the one or more univariates with a plurality of input non-correlated predictions associated with an input data record; and identifying, by the one or more processors, at least one missing data feature from a plurality of data features associated with the plurality of reference non-correlated predictions that is missing from the input data record based on the correlated prediction.

Example 2. The computer-implemented method of any of the preceding examples further comprising generating, using a predictive machine learning model, the plurality of reference non-correlated predictions for a plurality of reference data records.

Example 3. The computer-implemented method of any of the preceding examples further comprising: determining a first data feature, associated with a first prediction of the plurality of input non-correlated predictions, is assigned to the input data record; and modifying the first prediction and a second prediction of the plurality of input non-correlated predictions based on (i) the determination of the first data feature and (ii) a hierarchical relationship between the first data feature and a second data feature associated with the second prediction.

Example 4. The computer-implemented method of any of the preceding examples, wherein generating the simulation matrix further comprises generating the simulation matrix based on a Monte Carlo simulation.

Example 5. The computer-implemented method of any of the preceding examples further comprising generating a Cholesky decomposition matrix based on the correlation matrix.

Example 6. The computer-implemented method of any of the preceding examples, wherein generating the plurality of correlated simulation data records comprises generating a dot product of the Cholesky decomposition matrix and the plurality of simulation data records.

Example 7. The computer-implemented method of any of the preceding examples, wherein the plurality of co-occurrence values comprises a plurality of positive or negative values.

Example 8. A computing system comprising memory and one or more processors communicatively coupled to the memory, the one or more processors configured to: generate a correlation matrix based on a plurality of co-occurrence values associated with a plurality of reference non-correlated predictions; generate a simulation matrix comprising a plurality of simulation data records based on a number of simulation instances and the plurality of reference non-correlated predictions; generate a plurality of correlated simulation data records based on the correlation matrix and select ones of the plurality of simulation data records; generate one or more univariates based on the plurality of correlated simulation data records; generate a correlated prediction by comparing the one or more univariates with a plurality of input non-correlated predictions associated with an input data record; and identify at least one missing data feature from a plurality of data features associated with the plurality of reference non-correlated predictions that is missing from the input data record based on the correlated prediction.

Example 9. The computing system of any of the preceding examples, wherein the one or more processors are further configured to generate, using a predictive machine learning model, the plurality of reference non-correlated predictions for a plurality of reference data records.

Example 10. The computing system of any of the preceding examples, wherein the one or more processors are further configured to: determine a first data feature, associated with a first prediction of the plurality of input non-correlated predictions, is assigned to the input data record; and modify the first prediction and a second prediction of the plurality of input non-correlated predictions based on (i) the determination of the first data feature and (ii) a hierarchical relationship between the first data feature and a second data feature associated with the second prediction.

Example 11. The computing system of any of the preceding examples, wherein the one or more processors are further configured to generate the simulation matrix based on a Monte Carlo simulation.

Example 12. The computing system of any of the preceding examples, wherein the one or more processors are further configured to generate a Cholesky decomposition matrix based on the correlation matrix.

Example 13. The computing system of any of the preceding examples, wherein the one or more processors are further configured to generate a dot product of the Cholesky decomposition matrix and the plurality of simulation data records.

Example 14. The computing system of any of the preceding examples, wherein the plurality of co-occurrence values comprises a plurality of positive or negative values.

Example 15. One or more non-transitory computer-readable storage media including instructions that, when executed by one or more processors, cause the one or more processors to: generate a correlation matrix based on a plurality of co-occurrence values associated with a plurality of reference non-correlated predictions; generate a simulation matrix comprising a plurality of simulation data records based on a number of simulation instances and the plurality of reference non-correlated predictions; generate a plurality of correlated simulation data records based on the correlation matrix and select ones of the plurality of simulation data records; generate one or more univariates based on the plurality of correlated simulation data records; generate a correlated prediction by comparing the one or more univariates with a plurality of input non-correlated predictions associated with an input data record; and identify at least one missing data feature from a plurality of data features associated with the plurality of reference non-correlated predictions that is missing from the input data record based on the correlated prediction.

Example 16. The one or more non-transitory computer-readable storage media of any of the preceding examples further including instructions that, when executed by the one or more processors, cause the one or more processors to generate, using a predictive machine learning model, the plurality of reference non-correlated predictions for a plurality of reference data records.

Example 17. The one or more non-transitory computer-readable storage media of any of the preceding examples further including instructions that, when executed by the one or more processors, cause the one or more processors to: determine a first data feature, associated with a first prediction of the plurality of input non-correlated predictions, is assigned to the input data record; and modify the first prediction and a second prediction of the plurality of input non-correlated predictions based on (i) the determination of the first data feature and (ii) a hierarchical relationship between the first data feature and a second data feature associated with the second prediction.

Example 18. The one or more non-transitory computer-readable storage media of any of the preceding examples further including instructions that, when executed by the one or more processors, cause the one or more processors to generate the simulation matrix based on a Monte Carlo simulation.

Example 19. The one or more non-transitory computer-readable storage media of any of the preceding examples further including instructions that, when executed by the one or more processors, cause the one or more processors to generate a Cholesky decomposition matrix based on the correlation matrix.

Example 20. The one or more non-transitory computer-readable storage media of any of the preceding examples further including instructions that, when executed by the one or more processors, cause the one or more processors to generate a dot product of the Cholesky decomposition matrix and the plurality of simulation data records.

Example 21. The one or more non-transitory computer-readable storage media of any of the preceding examples, wherein the plurality of co-occurrence values comprises a plurality of positive or negative values.

1. A computer-implemented method comprising:
generating, by one or more processors, a correlation matrix based on a plurality of co-occurrence values associated with a plurality of reference non-correlated predictions;
generating, by the one or more processors, a simulation matrix comprising a plurality of simulation data records based on a number of simulation instances and the plurality of reference non-correlated predictions;
generating, by the one or more processors, a plurality of correlated simulation data records based on the correlation matrix and select ones of the plurality of simulation data records;
generating, by the one or more processors, one or more univariates based on the plurality of correlated simulation data records;
generating, by the one or more processors, a correlated prediction by comparing the one or more univariates with a plurality of input non-correlated predictions associated with an input data record; and
identifying, by the one or more processors, at least one missing data feature from a plurality of data features associated with the plurality of reference non-correlated predictions that is missing from the input data record based on the correlated prediction.

2. The computer-implemented method of claim 1 further comprising:
generating, using a predictive machine learning model, the plurality of reference non-correlated predictions for a plurality of reference data records.

3. The computer-implemented method of claim 1 further comprising:
determining a first data feature, associated with a first prediction of the plurality of input non-correlated predictions, is assigned to the input data record; and
modifying the first prediction and a second prediction of the plurality of input non-correlated predictions based on (i) the determination of the first data feature and (ii) a hierarchical relationship between the first data feature and a second data feature associated with the second prediction.

4. The computer-implemented method of claim 1, wherein generating the simulation matrix further comprises generating the simulation matrix based on a Monte Carlo simulation.

5. The computer-implemented method of claim 1 further comprising generating a Cholesky decomposition matrix based on the correlation matrix.

6. The computer-implemented method of claim 5 wherein generating the plurality of correlated simulation data records comprises generating a dot product of the Cholesky decomposition matrix and the plurality of simulation data records.

7. The computer-implemented method of claim 1 wherein the plurality of co-occurrence values comprises a plurality of positive or negative values.

8. A computing system comprising memory and one or more processors communicatively coupled to the memory, the one or more processors configured to:
generate a correlation matrix based on a plurality of co-occurrence values associated with a plurality of reference non-correlated predictions;
generate a simulation matrix comprising a plurality of simulation data records based on a number of simulation instances and the plurality of reference non-correlated predictions;
generate a plurality of correlated simulation data records based on the correlation matrix and select ones of the plurality of simulation data records;
generate one or more univariates based on the plurality of correlated simulation data records;
generate a correlated prediction by comparing the one or more univariates with a plurality of input non-correlated predictions associated with an input data record; and
identify at least one missing data feature from a plurality of data features associated with the plurality of reference non-correlated predictions that is missing from the input data record based on the correlated prediction.

9. The computing system of claim 8, wherein the one or more processors are further configured to generate, using a predictive machine learning model, the plurality of reference non-correlated predictions for a plurality of reference data records.

10. The computing system of claim 8, wherein the one or more processors are further configured to:
determine a first data feature, associated with a first prediction of the plurality of input non-correlated predictions, is assigned to the input data record; and
modify the first prediction and a second prediction of the plurality of input non-correlated predictions based on (i) the determination of the first data feature and (ii) a hierarchical relationship between the first data feature and a second data feature associated with the second prediction.

11. The computing system of claim 8, wherein the one or more processors are further configured to generate the simulation matrix based on a Monte Carlo simulation.

12. The computing system of claim 8, wherein the one or more processors are further configured to generate a Cholesky decomposition matrix based on the correlation matrix.

13. The computing system of claim 12, wherein the one or more processors are further configured to generate a dot product of the Cholesky decomposition matrix and the plurality of simulation data records.

14. The computing system of claim 8, wherein the plurality of co-occurrence values comprises a plurality of positive or negative values.

15. One or more non-transitory computer-readable storage media including instructions that, when executed by one or more processors, cause the one or more processors to:
generate a correlation matrix based on a plurality of co-occurrence values associated with a plurality of reference non-correlated predictions;
generate a simulation matrix comprising a plurality of simulation data records based on a number of simulation instances and the plurality of reference non-correlated predictions;
generate a plurality of correlated simulation data records based on the correlation matrix and select ones of the plurality of simulation data records;
generate one or more univariates based on the plurality of correlated simulation data records;
generate a correlated prediction by comparing the one or more univariates with a plurality of input non-correlated predictions associated with an input data record; and
identify at least one missing data feature from a plurality of data features associated with the plurality of reference non-correlated predictions is that missing from the input data record based on the correlated prediction.

16. The one or more non-transitory computer-readable storage media of claim 15 further including instructions that, when executed by the one or more processors, cause the one or more processors to generate, using a predictive machine learning model, the plurality of reference non-correlated predictions for a plurality of reference data records.

17. The one or more non-transitory computer-readable storage media of claim 15 further including instructions that, when executed by the one or more processors, cause the one or more processors to:
determine a first data feature, associated with a first prediction of the plurality of input non-correlated predictions, is assigned to the input data record; and
modify the first prediction and a second prediction of the plurality of input non-correlated predictions based on (i) the determination of the first data feature and (ii) a hierarchical relationship between the first data feature and a second data feature associated with the second prediction.

18. The one or more non-transitory computer-readable storage media of claim 15 further including instructions that, when executed by the one or more processors, cause the one or more processors to generate the simulation matrix based on a Monte Carlo simulation.

19. The one or more non-transitory computer-readable storage media of claim 15 further including instructions that, when executed by the one or more processors, cause the one or more processors to generate a Cholesky decomposition matrix based on the correlation matrix.

20. The one or more non-transitory computer-readable storage media of claim 19 further including instructions that, when executed by the one or more processors, cause the one or more processors to generate a dot product of the Cholesky decomposition matrix and the plurality of simulation data records.
